(19) United States
(12) Patent Application Publication
(10) Pub. No.: US 2024/0119589 A1
(43) Pub. Date: Apr. 11, 2024

(54) METHOD AND DEVICE FOR DETECTING A PRESENCE OF A FLUORESCENCE PATTERN ON AN IMMUNOFLUORESCENCE IMAGE OF A BIOLOGICAL CELL SUBSTRATE

(71) Applicant: EUROIMMUN Medizinische Labordiagnostika AG, Lobeck (DE)

(72) Inventors: Jens KRAUTH, Lübeck (DE); Jens HOCKE, Lübeck (DE); Stefan GERLACH, Lübeck (DE); Christopher KRAUSE, Lübeck (DE); Melanie HAHN, Lübeck (DE); Jörn VOIGT, Lübeck (DE)

(73) Assignee: EUROIMMUN Medizinische Labordiagnostika AG, Lübeck (DE)

(21) Appl. No.: 18/371,979

(22) Filed: Sep. 22, 2023

(30) Foreign Application Priority Data
Sep. 30, 2022 (EP) 22199099.7

(51) Int. Cl. G06T 7/00; G01N 33/58; G06T 7/73; G06V 10/82; G06V 20/69

(52) U.S. Cl. CPC G06T 7/0012; G01N 33/582; G06T 7/73; G06V 10/82; G06V 20/695; G06V 20/698; G06T 2207/10056; G06T 2207/10064; G06T 2207/20084; G06T 2207/30024; G06V 2201/03

(57) ABSTRACT

A method is proposed for detecting a presence of a fluorescence pattern on an immunofluorescence image of a biological cell substrate, comprising the following steps: incubating the cell substrate with a liquid patient sample, which potentially comprises primary antibodies, and furthermore with secondary antibodies, which are marked using a fluorescence stain, irradiating the cell substrate using excitation radiation and capturing the immunofluorescence image, determining respective items of location information, which indicate respective locations of respective relevant subsections of the cell substrate in the fluorescence image, and determining respective first partial confidence measures of respective presences of the fluorescence pattern on the respective subsections by means of a first neural network on the basis of the overall fluorescence image, extracting respective image subsections, which correspond to the respective subsections of the cell substrate, from the fluorescence image on the basis of the items of location information, determining respective second partial confidence measures of respective presences of the fluorescence pattern on the respective subsections by means of a second neural network on the basis of the respective image subsections, determining a confidence measure of the presence of the fluorescence pattern in the fluorescence image on the basis of the first partial confidence measures and the second partial confidence measures.

The invention relates to a method and a device for detecting a presence of a fluorescence pattern on an immunofluorescence image of a biological cell substrate. The invention furthermore relates to a digital image processing method, a computer program product, a data carrier signal, a computing unit, and a data network device.

Immunofluorescence images of biological cell substrates can be captured by image capture units such as microscopes. Information with respect to a presence of a fluorescence pattern to be expected on a fluorescence image can be advantageous for the purpose of assisting a diagnostic question.

Immunofluorescence microscopy or indirect immunofluorescence microscopy is an in vitro test for a determination of a presence of human antibodies against specific antigens, in order to be able to answer or assess a diagnostic question. Such antigens are, for example, in specific areas of biological cell substrates. A cell substrate which is incubated using a patient sample in the form of blood or diluted blood or blood serum or diluted blood serum is thus used as the substrate. The patient sample thus potentially has specific primary antibodies, which can be the expression of a presence of a disease of the patient. Such primary or specific antibodies can then bind to antigens of the substrate. Such bound primary antibodies can then be marked in that in a further incubation step, so-called secondary antibodies, preferably antihuman antibodies, bind to the bound primary antibodies and can later be made visible in that the secondary antibodies are marked using a fluorescence stain. Such a fluorescence stain is preferably a green fluorescence stain, in particular the fluorescence stain FITC. Such binding of a primary antibody jointly with a fluorescence-marked secondary antibody can then be made visible later in that the substrate is irradiated using excitation light of a specific wavelength and the bound fluorescence stains are thus excited to emit fluorescence radiation.

Depending on the diagnostic question, a presence of one or very specific fluorescence pattern types on specific substrate areas can be important. The object thus results of detecting a presence of a potential fluorescence pattern in a fluorescence image of immunofluorescence microscopy via digital image processing in the course of immunofluorescence microscopy in a cell substrate incubated in the above-described manner.

A biological cell substrate can in this case in particular be a cell smear of cell lines, a colony of cell lines, and/or biological tissue having biological tissue cells, in particular from a tissue section.

In the indirect immunofluorescence test (IIFT), freezing-fixed tissue sections of the substrate primate pancreas are preferably used to detect auto antibodies against pancreas islets (islet cell antibodies=ICA). The pancreas can be divided into an exocrine and an endocrine area. Antibodies against islet cells only react with the endocrine part of the organ, the islets of Langerhans, also called “islets”, and are directed against the insulin-producing beta cells. The 65 kDa isoform of the enzyme glutamate decarboxylase (GAD65), the tyrosine phosphatase-homologous IA-2 proteins (IA-2α und IA-2β), the cation transporter ZnT8, insulin, and the insulin precursor proinsulin were identified as antigens.

The resulting destruction of the beta cells results in the lack of insulin and thus in diabetes mellitus type 1. In 70 to 80% of the type 1 diabetics, it was possible to detect islet cell antibodies at the point in time of clinical manifestation. They are therefore an important distinguishing feature between diabetes mellitus type 1 and diabetes mellitus type 2 (adult-onset diabetes).

With respect to a special form of progress of diabetes mellitus type 1, latent autoimmune diabetes in adults (LADA), islet cell antibodies are also used as a criterion for the need for insulin of the patients. LADA is present in 3 to 12% of the patients having phenotypical diabetes mellitus type 2, which is characterized by insulin resistance and disturbed insulin secretion of the P cells.

In 90% of the patients, islet cell antibodies are already detectable years before the diabetes manifestation. They are therefore considered a marker of the so-called prediabetic phase. Early identification of the persons having increased risk of disease can thus be ensured. Under certain circumstances, onset of the disease may thus be prevented by suitable interventions. The titre for islet cell antibodies decreases again with progress of the disease. A very high concentration of auto antibodies against islet cells (GAD65) can be an indication of stiff person syndrome, cerebellar degeneration, and limbic encephalitis.

In the indirect immunofluorescence test, the islets of Langerhans of the pancreatic tissue display a fine-spotted, smooth to grainy fluorescence in the case of a positive result.

Furthermore, a substrate can preferably be used in the indirect immunofluorescence test (IIFT), in which multiple Crithidia luciliae cells are fixed on the substrate. Such a substrate is in particular a cell smear. Such a fixation can be performed, for example, by means of ethanol. The substrate can then be used for detecting bonding of autoantibodies of a patient sample to double-stranded deoxyribonucleic acid (DNA). The detection of autoantibodies against deoxyribonucleic acid (DNA) is of crucial importance, for example, for the diagnosis of SLE (synonym: Lupus erythematodes disseminatus). It is necessary for this purpose to fundamentally distinguish two types: antibodies against dsDNA and antibodies against single-stranded, denatured DNA (single-stranded DNA, ssDNA). Antibodies against dsDNA react with epitopes which are present in the deoxyribose phosphate framework of the DNA. In contrast, antibodies against ssDNA predominantly bind to epitopes from the area of the purine or pyrimidine bases. However, they can also identify epitopes of the deoxyribose phosphate framework. Anti-dsDNA antibodies are found almost exclusively in the case of SLE. The prevalence is 20% to 90%, depending on the detection method and disease activity. Anti-dsDNA antibodies are sometimes also detected in patients having other autoimmune diseases and infections and also in rare cases in clinically healthy persons. The latter develop an SLE within 5 years after the anti-dsDNA initial detection in 85% of the cases. However, an SLE cannot be excluded entirely if no antibodies against dsDNA are present. SLE is a systemic autoimmune disease from the group of connective tissue diseases. The diagnosis is directed to the 11 criteria of the American College of Rheumatology (ACR), which were modified in 1997. If 4 of the 11 criteria are present, the diagnosis of an SLE can be made with 80 to 90% certainty. Indirect immunofluorescence can thus be an in vitro test for the determination of human antibodies against dsDNA. For example, so-called BIOCHIPs can be used as the substrate, which are coated using Crithidia luciliae smears. These are incubated, for example, with diluted patient samples. In the event of positive reactions, specific antibodies bind to the antigens. Bound antibodies (IgG) are stained in a second incubation step, for example, using fluorescein-marked antihuman antibodies and made visible in the fluorescence microscope.

The object of the present invention is to provide an automated method for analysis of immunofluorescence image of a biological cell substrate, which can detect a presence of a fluorescence pattern on the cell substrate.

The object according to the invention is achieved by the method according to the invention according to Claim 1.

According to the invention, the method comprises various steps.

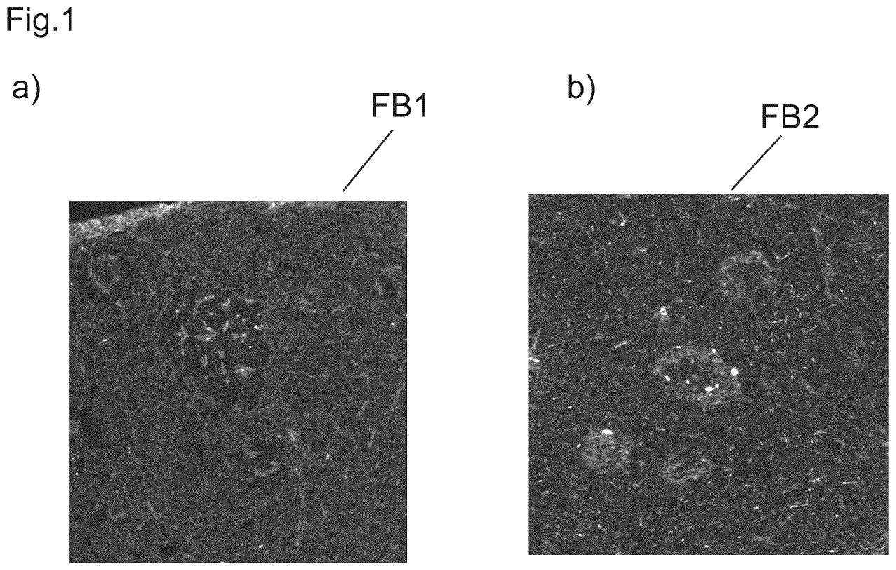

FIG. 1A shows an exemplary fluorescence image FB1, in this example a tissue section of a pancreas. In the example of the fluorescence image FB1, a fluorescence pattern to be expected is not present. FIG. 1B shows a further fluorescence image FB2, also in this case a pancreas, wherein a fluorescence pattern to be expected is present here.

For the example of a substrate in the form of a pancreas section, the focus is to be directed to a specific or relevant subsection, which are the so-called islets.

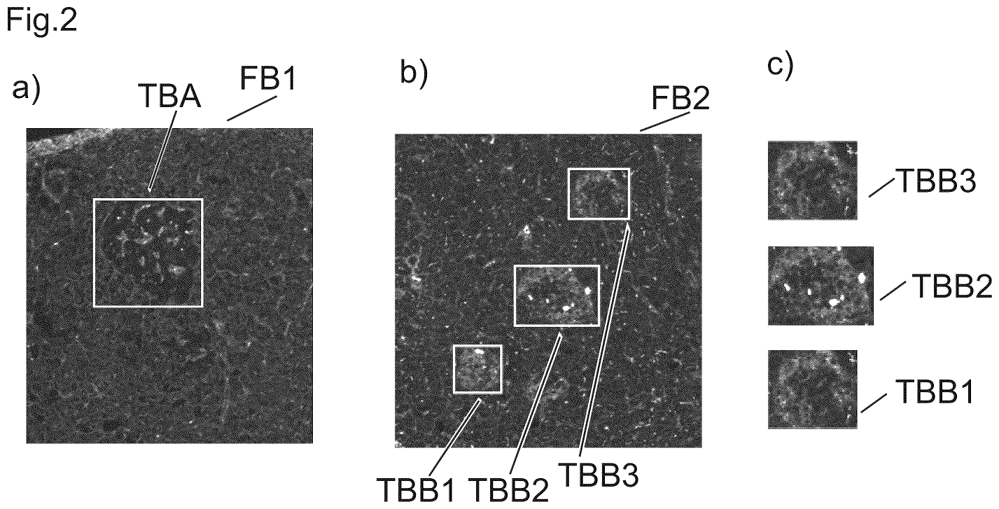

FIG. 2A shows for this purpose for the fluorescence image FB1 an area TBA as an image subsection, which essentially comprises an islet. No fluorescence pattern to be expected is present in the area TBA. FIG. 2B shows for this purpose for the fluorescence image FB2 partial image areas TBB1, TBB2, TBB3 having corresponding islets, on which a fluorescence pattern to be expected is present. These image subsections TBB1, . . . , TBB3 are shown once again separately and enlarged in FIG. 2C.

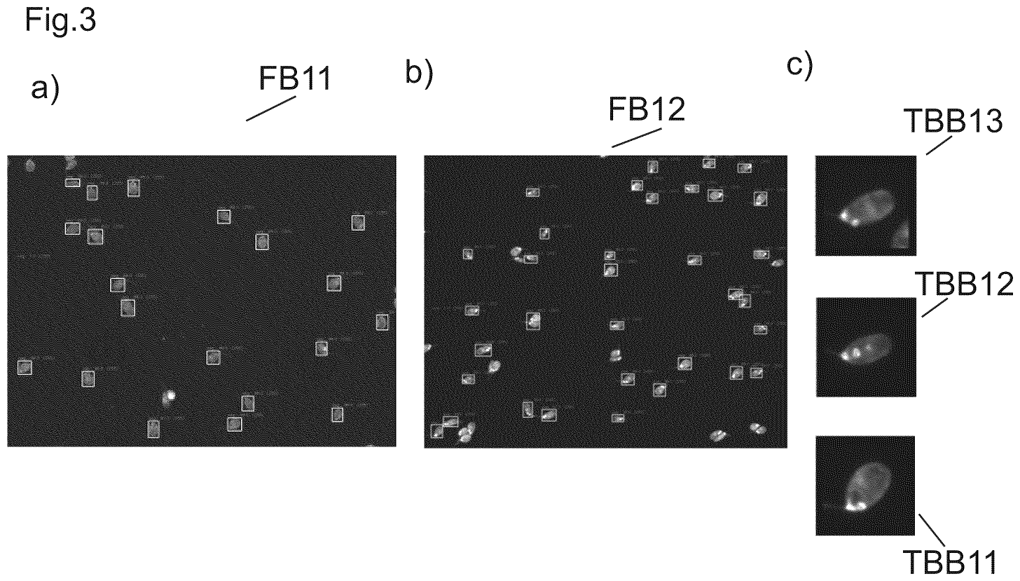

FIG. 3A shows for the example of a cell substrate in the form of a cell smear of Crithidia luciliae cells a fluorescence image FB11, in which essentially no fluorescence pattern to be expected is present. FIG. 3B shows an example of a fluorescence image with a cell substrate in the form of a cell smear having Crithidia luciliae cells, in which a fluorescence pattern to be expected is present as the image FB12. Relevant subsections or image subsections are indicated in this case in the fluorescence image FB12 by rectangles. The relevant subsections in particular comprise a kinetoplast of a Crithidia luciliae cell here.

FIG. 3C shows by way of example image subsections TBB11, TBB12, TBB13 from the fluorescence image FB12, in which a fluorescence pattern to be expected is present in the relevant areas in the form of the Crithidia luciliae cells. This is in particular a light of the kinetoplast.

According to the invention, after the fluorescence image is captured, respective locating information is determined, which indicates respective locations of respective relevant subsections of the cell substrate in the fluorescence image. For the example of the pancreas, the relevant subsections are simply the so-called islets, since a fluorescence pattern to be expected has to be detected thereon or it has to be determined whether the fluorescence pattern to be expected is not present. Such locating information can be determined, for example, in the form of a rectangle, as shown in FIG. 2B, for a respective relevant subsection.

Furthermore, according to the invention, respective first partial confidence measures of respective presences of the fluorescence pattern on the respective subsections are determined on the basis of the overall fluorescence image. Such subsections correspond, for example, to the image subsections TBB1, TBB2, TBB3 from FIG. 2B.

The location information and the respective first partial confidence measures are determined using a first neural network on the basis of the overall fluorescence image. In particular, this determination of the location information and the first partial confidence measures is carried out simultaneously by the first neural network, so that the first neural network is in particular a combined neural network for both tasks of this determination.

According to the invention, the respective image subsections which correspond to the respective subsections of the cell substrate are extracted from the fluorescence image. This is carried out on the basis of the previously obtained location information. Such image subsections are shown by way of example in FIG. 2C as image subsections TBB1, TBB2, TBB3.

Furthermore, according to the invention, respective second partial confidence measures with respect to respective presences of the fluorescence pattern on the respective subsections are determined using a second neural network on the basis of the respective image subsections. Thus, for example, the image subsections TBB1, TBB2, TBB3 from FIG. 2C are analysed using a second neural network. In particular, the respective second partial confidence measures are determined using the second neural network such that each of the respective image subsections is analysed or processed separately as such by the second neural network in order to determine a respective second partial confidence measure.

According to the invention, a confidence measure of the presence of the fluorescence pattern in the fluorescence image is finally determined on the basis of the first partial confidence measures and the second partial confidence measures.

Possible advantages of the invention will now be explained in more detail hereinafter.

The fundamental object of detecting a presence of a fluorescence pattern on a large overall fluorescence image could in principle be performed by a single neural network in that such large overall images having corresponding ground truth information “positive” or“negative” with respect to the presence of the fluorescence pattern are analysed in a training phase by such a single neural network in order to then determine such a class assignment to the classes “positive” or “negative”. In such a procedure, this single neural network would have to allow a large amount of image information from an overall fluorescence image to be incorporated and taken into consideration. This would require a high level of complexity of the network and thus also a large amount of computing operations, so that the corresponding computing effort can represent a challenge.

Furthermore, it has to be noted that neural networks for analysing large overall images can extract those image features and possibly classify them as relevant, which are present in the overall image but can be irrelevant for the actual classification task of the neural network. In order to guide the attention and the image features to be learned of the neural network in a targeted manner only onto the image structures which are actually relevant, the method proposed here suggests itself, in order to initially directly identify each of the relevant structures of the overall fluorescence image with respect to their location and also classify each of them in a first step using a first neural network and then to additionally classify each of the corresponding image subsections of the relevant structures as such by way of a second neural network in a second step. In this case, by ascertaining the classification results or the first partial confidence measures from the first step in combination with ascertaining the classification results or the second partial confidence measures from the second step and combining these results in the final confidence measure of the presence of the fluorescence pattern in the overall fluorescence image, a high level of validity of the final confidence measure results. A further advantage results in that it is possible via the partial confidence measures of the subsections or the image subsections to allow the classification results from the two steps or from the two neural networks to be integrated in a controlled manner in the final confidence measure. Good model interpretability results in this way with respect to the function of the two neural networks in the method. Due to the ascertainment of the relevant subsections and their corresponding image subsections as individual elements, the number of the partial results available in the method, for example, in the form of partial confidence measures, results automatically, wherein such information or number information is not present in an overall image approach, since then there is only an overall output of a single confidence measure with respect to the overall image, preferably as the statement “positive” or “negative”.

In other words: The method according to the invention deviates explicitly from an approach for analysing an overall fluorescence image using only one neural network in that initially the first neural network determines items of location information which indicate relevant subsections of the cell substrate. The second neural network then only has to process such an image subsection separately as such in each case in order to detect a possible presence of the fluorescence pattern in the respective image subsection and ascertain a corresponding partial confidence measure.

The second neural network can thus be significantly reduced in its complexity in relation to a solution in which a single neural network has to ascertain the confidence measure of the presence of the fluorescence pattern for the overall fluorescence image.

The second neural network can thus be trained on significantly smaller image subsections and only has to analyse those types of partial images which actually represent relevant subsections of a cell substrate.

The second neural network can therefore ascertain significantly more valid partial confidence measures with respect to presences of the fluorescence pattern on the respective partial images or subsections. The second neural network can therefore also determine a more valid confidence measure with respect to a presence of the fluorescence pattern in the fluorescence image.

The method according to the invention is furthermore advantageous since the first neural network both determines the relevant subsections using the location information and determines the first partial confidence measures, in particular simultaneously. The first neural network is thus in particular one which was trained for simultaneously determining the first partial confidence measures and determining the location information. Such networks can be particularly efficient with respect to computing complexity and required computing memory in relation to other networks. Such networks are implementable in particular as so-called “one-shot detectors”.

Furthermore, the method according to the invention is advantageous since the confidence measure is determined in consideration or on the basis of the first partial confidence measures and the second partial confidence measures, so that such information with respect to the partial confidence measures can be incorporated from both neural networks, in order to determine a particularly reliable confidence measure of the presence of the fluorescence pattern in the fluorescence image.

The confidence measure is preferably output.

Preferably, weighted partial confidence measures are determined via weighting of the first partial confidence measures and the second partial confidence measures, and the confidence measure is determined on the basis of the weighted partial confidence measures.

The confidence measure is preferably determined via application of a threshold value to the weighted partial confidence measures.

The method preferably furthermore comprises the following steps: Determining respective presence confidence measures which indicate for the respective locations of the respective relevant subsections to which degree actually relevant subsections of the cell substrate are present at the respective locations using the first neural network on the basis of the overall fluorescence image, determining whether the items of location information indicate a set of multiple overlapping image subsections, retaining the location information of a specific image subsection from the set of overlapping image subsections on the basis of the presence confidence measures of the overlapping image subsections and discarding the items of location information of the other image subsections from the set of overlapping image subsections, and extracting respective image subsections from the fluorescence image on the basis of the remaining items of location information.

Furthermore, the method in particular comprises the following steps: determining a set of image subsections which have a presence of the fluorescence pattern, and ascertaining a brightness value of the presence of the fluorescence pattern on the immunofluorescence image on the basis of the set of image subsections which have a presence of the fluorescence pattern.

Furthermore, a method for digital image processing is proposed which comprises the following steps: providing an immunofluorescence image which represents a staining of a biological cell substrate by a fluorescence stain, determining respective items of location information which indicate respective locations of respective relevant subsections of the cell substrate in the fluorescence image, and determining respective first partial confidence measures of respective presences of the fluorescence pattern on the respective subsections using a first neural network on the basis of the overall fluorescence image, extracting respective image subsections which correspond to the respective subsections of the cell substrate on the basis of the location information, determining respective second partial confidence measures of respective presences of the fluorescence pattern on the respective subsections using a second neural network on the basis of the respective image subsections, and determining a confidence measure of the presence of the presence of the fluorescence pattern in the fluorescence image on the basis of the first partial confidence measures and the second partial confidence measures.

Furthermore, a computer program product is proposed comprising commands which, upon the execution of the program by a computer, prompt it to carry out the method for digital image processing.

Furthermore, a data carrier signal which transmits the computer program product is proposed.

Furthermore, a device is proposed for detecting a fluorescence pattern on an immunofluorescence image of a biological cell substrate, comprising a holding device for an object carrier having the cell substrate, which was incubated with a patient sample, including the autoantibodies, and furthermore with secondary antibodies, which are each marked with a fluorescent material. The device furthermore comprises at least one image capture unit for capturing a fluorescence image of the cell substrate. Furthermore, the device comprises at least one computing unit, which is designed to carry out the following steps: determining respective items of location information, which index respective locations of respective relevant subsections of the cell substrate in the fluorescence image, and determining respective first partial confidence measures of respective presences of the fluorescence pattern on the respective subsections using a first neural network on the basis of the overall fluorescence image, extracting respective image subsections which correspond to the respective subsections of the cell substrate on the basis of the items of location information, determining respective second partial confidence measures of respective presences of the fluorescence pattern on the respective subsections using a second neural network on the basis of the respective image subsections, and determining a confidence measure of the presence of the fluorescence pattern in the fluorescence image on the basis of the first partial confidence measures and the second partial confidence measures.

Furthermore, a computing unit is proposed which is designed to execute the following steps in the course of digital image processing: accepting an immunofluorescence image which represents staining of a biological substrate by a fluorescent stain, determining respective items of location information which indicate respective locations of respective relevant subsections of the cell substrate in the fluorescence image, and determining respective first partial confidence measures of respective presences of the fluorescence pattern on the respective subsections using a first neural network on the basis of the overall fluorescence image, extracting respective image subsections which correspond to the respective subsections of the cell substrate on the basis of the items of location information, determining respective second partial confidence measures of respective presences of the fluorescence pattern on the respective subsections using a second neural network on the basis of the respective image subsections, and determining a confidence measure of the presence of the fluorescence pattern in the fluorescence image on the basis of the first partial confidence measures and the second partial confidence measures.

Furthermore, a data network device is proposed, comprising at least one data interface for accepting a fluorescence image which represents staining of a cell substrate by a fluorescence stain. The data network device furthermore comprises at least one computing unit which is designed in the course of digital image processing to execute the following steps: determining respective items of location information which indicate respective locations of respective relevant subsections of the cell substrate in the fluorescence image, and determining respective first partial confidence measures of respective presences of the fluorescence pattern on the respective subsections using a first neural network on the basis of the overall fluorescence image, extracting respective image subsections, which correspond to the respective subsections of the cell substrate, on the basis of the items of location information, determining respective second partial confidence measures of respective presences of the fluorescence pattern on the respective subsections using a second neural network on the basis of the respective image subsections, and determining a confidence measure of the presence of the fluorescence pattern in the fluorescence image on the basis of the first partial confidence measures and the second partial confidence measures.

The invention is explained hereinafter in more detail on the basis of special embodiments without restriction of the general concept of the invention on the basis of the figures. In the figures:

FIGS. 1 and 2 show immunofluorescence images and partial images of a first biological cell substrate,

FIG. 3 shows immunofluorescence images and partial images of a second biological cell substrate,

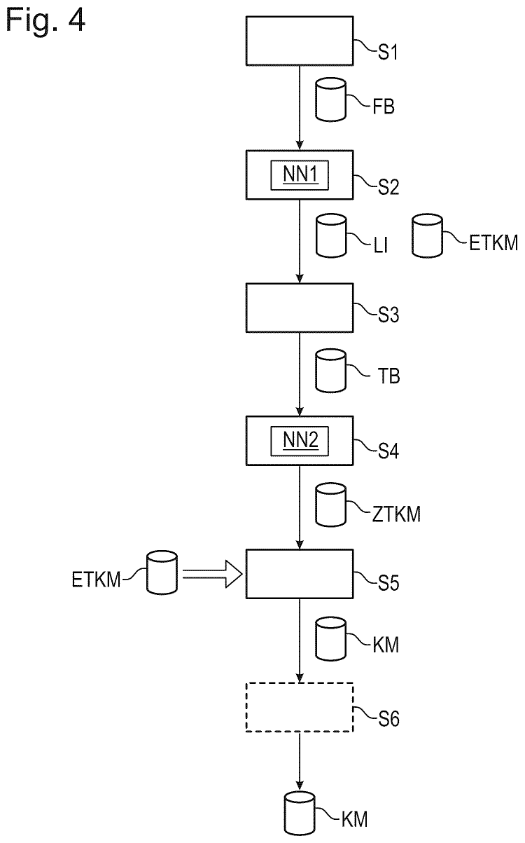

FIG. 4 shows steps of a preferred embodiment of the method according to the invention,

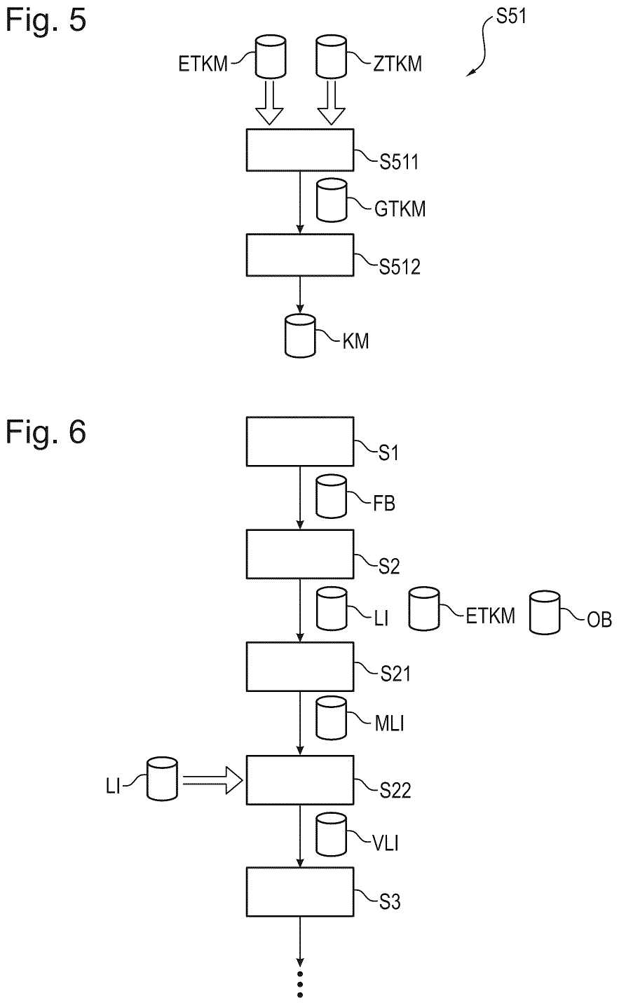

FIG. 5 shows preferred steps for determining the confidence measure with weighting of partial confidence measures,

FIG. 6 shows the steps of the method according to the invention with respect to a further preferred embodiment,

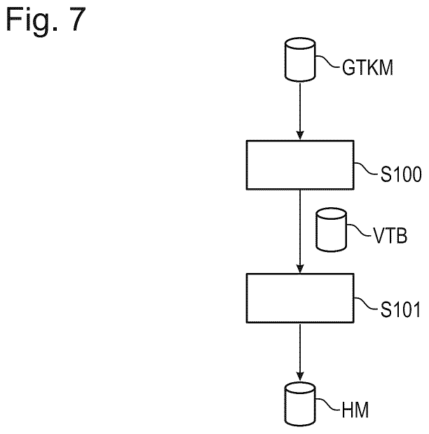

FIG. 7 shows steps for determining a brightness measure of the presence of the fluorescence pattern,

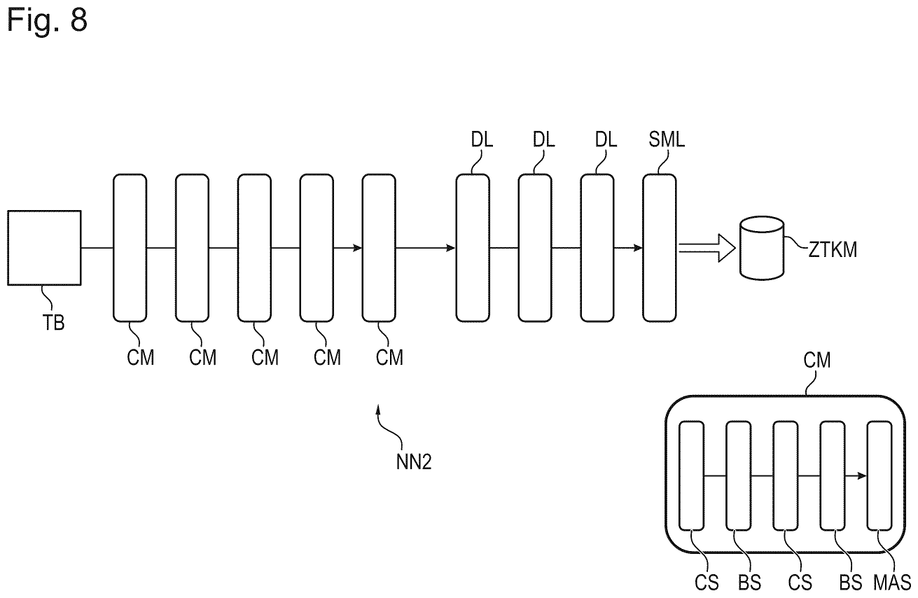

FIG. 8 shows a preferred embodiment of a second neural network,

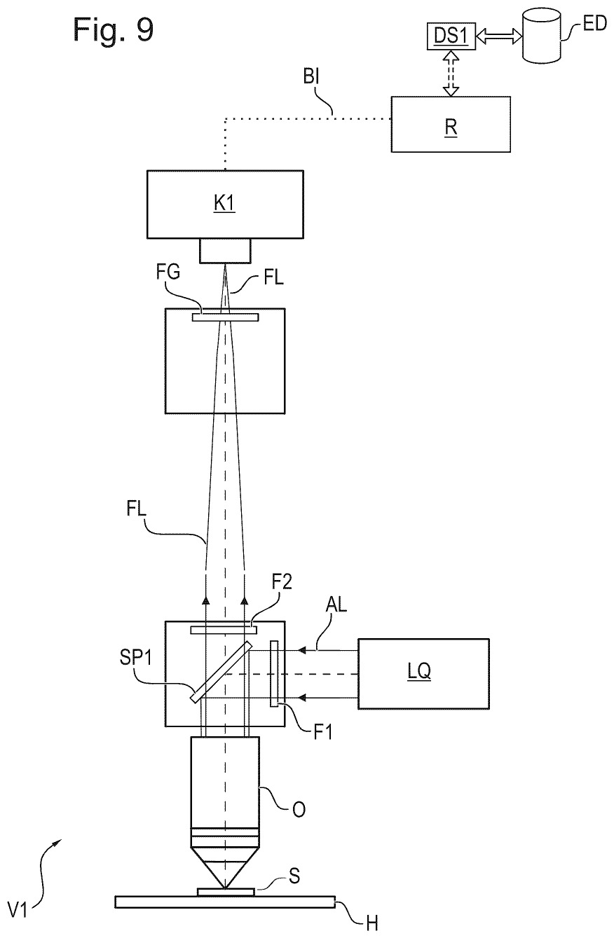

FIG. 9 shows a preferred embodiment of a device according to the invention,

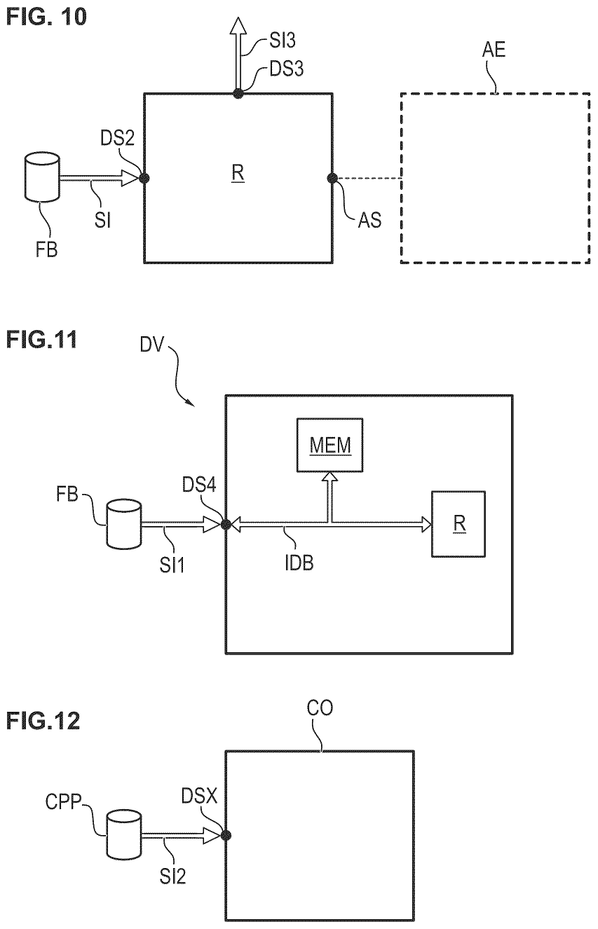

FIG. 10 shows a preferred embodiment of a computing unit according to the invention,

FIG. 11 shows a preferred embodiment of a data network device according to the invention,

FIG. 12 shows an illustration of a computer program product and a data carrier signal,

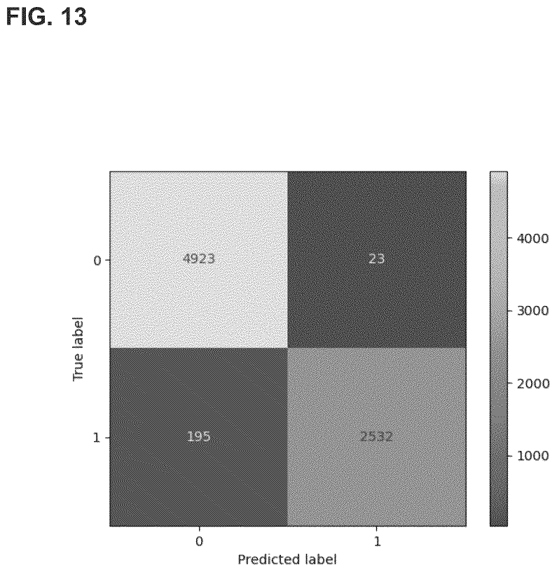

FIG. 13 shows a result table of experimental results.

FIG. 4 shows steps of a preferred embodiment of the method according to the invention. In a step S1, the cell substrate is incubated with a liquid patient sample, which potentially comprises potential primary antibodies, and furthermore with secondary antibodies which are marked using a fluorescent stain. Furthermore, in step S1, the cell substrate is irradiated using excitation radiation and the immunofluorescence image is captured.

The immunofluorescence image can be provided as a data element FB by step S1.

Such an immunofluorescence image is shown by way of example as an image FB1 in FIG. 1A. In this case, the immunofluorescence is not present in the relevant cell areas, here the eyelets, of the cell substrate, here in the form of a pancreas section. Such an immunofluorescence is present in the relevant subsections in the immunofluorescence image FB2 of FIG. 1B.

FIG. 2A shows the immunofluorescence image FB1 once again with an indicated subsection or an image subsection TBA, within which a relevant subsection of the cell substrate is located. In comparison to subsections or image subsections TBB1, TBB2, TBB3 of the fluorescence image FB2 from FIG. 2B, it is apparent that in the immunofluorescence image FB1, a presence of a fluorescence pattern to be expected is not provided, but this presence of the fluorescence pattern is provided in the image subsections of the immunofluorescence image FB2. The fluorescence patterns of the respective image subsections TBB1, TBB2, TBB3 can be seen once again in an enlarged illustration in FIG. 2C.

In the meaning of this application, a partial image can also be designated as an image subsection.

According to FIG. 4, in step S2, respective items of location information are determined, which indicate respective locations of respective relevant subsections of the cell substrate in the fluorescence image. This is carried out using a neural network NN1, which can be designated as a first neural network.

Furthermore, in step S2, respective first partial confidence measures of respective presences of the fluorescence pattern on the respective subsections or the respective relevant subsections are determined, in particular simultaneously, using the neural network NN1 on the basis of the overall fluorescence image. This thus takes place in particular in combined or simultaneous processing.

The ascertained items of location information can be provided as a combined data element LI. The first partial confidence measures can be provided as a data element ETKM.

An item of location information preferably indicates a position of a subsection in a fluorescence image, furthermore an item of width information and furthermore an item of height information of the subsection. The item of location information can thus preferably indicate a rectangle at a specific position of the fluorescence image and the height and the width of the rectangle.

Such an item of location information for a subsection or an image subsection can be provided as a vector n,

$$\vec{n} = [ \begin{matrix}n 1 \\ n 2 \\ n 3 \\ n 4\end{matrix} ]$$

wherein the entry n1 can indicate a X position and the entry n2 can indicate a Y position of a rectangle in the fluorescence image and furthermore the entry n3 can indicate a width of the rectangle and the entry n4 can indicate a height of the rectangle.

A first partial confidence measure ETKM for a subsection, for example, a subsection TBB1 from FIG. 2C, can be given, for example, by a vector o

$$\vec{o} = [ \begin{matrix}o 1 \\ o 2\end{matrix} ]$$

wherein the entry o1 indicates a probability that the image subsection belongs to a class of a positive fluorescence of the pattern and wherein the entry o2 indicates a probability that the subsection belongs to a class of a negative fluorescence of the pattern or an absence of the fluorescence pattern.

The items of location information can thus be a respective vector n per image subsection and the first partial confidence measures can thus be a respective vector o per image subsection.

The first neural network is preferably a so-called object detection network. The first neural network is particularly preferably an object detection network of the type of a so-called one-shot detector. In particular, the first neural network is a so-called YOLO network.

Such a network can be trained, for example, in that overall fluorescence images are presented to an input layer of the neural network, wherein furthermore items of location information of respective subsections of the cell substrate are provided as ground truth information and also items of ground truth information with respect to a class membership of a corresponding image subsection of the subsection for the classes “positive fluorescence” or “negative fluorescence”.

The first neural network NN1 preferably furthermore ascertains a further item of information OB on the basis of the overall fluorescence image, also shown in FIG. 6. This is an item of information OB which is a presence confidence measure per location or per location information, which indicates for a respective location of a respective relevant subsection to which degree a relevant subsection of the cell substrate is actually present at the respective location. This item of information OB can be output for a respective image subsection via a respective scalar value c.

The first neural network thus preferably outputs one tuple {c, \vec{o}, \vec{n}} per detected relevant image subsection with index k=1 . . . K. The training of the neural network can thus in particular be carried out with provision of overall fluorescence images as ground truth information in the form of corresponding tuples {c, \vec{o}, \vec{n}} per subsection.

Detailed structures of such a first neural network for the special embodiment of a Yolo network can be found in J. Redmon, S. Divvala, R. Girshick and A. Farhadi, “You Only Look Once: Unified, Real-Time Object Detection,” 2016 IEEE Conference on Computer Vision and Pattern Recognition (CVPR), 2016, pp. 779-788, doi: 10.1109/CVPR.2016.91.

The confidence measure c is designated therein as the so-called “confidence”. The vector n is given therein by values of image position x, y and also width b and height h. The class probabilities o1 and o2 are given therein as so-called conditional class probabilities “C”.

In a step S3, respective image subsections, which correspond to the respective subsections of the cell substrate, are extracted from the fluorescence image on the basis of the location information. These are, for example, the image subsections from FIG. 2C. These image subsections can be provided as a combined data element TB. An extracted image subsection is preferably identical to the corresponding subsection which is indicated by corresponding location information. In particular, an image subsection can have a certain deviation from the subsection, in particular in the edge area.

In a step S4, furthermore the respective second partial confidence measures of respective presences of the fluorescence pattern on the respective subsections are determined using a second neural network NN2 on the basis of the respective corresponding image subsections.

Step S4 can provide these second partial confidence measures as a data element ZTKM.

The second neural network preferably outputs a vector d having a class probability for the respective classes “fluorescence positive” or “fluorescence negative” per image subsection. In particular, the vector d can thus be a vector

$$\vec{d} = [ \begin{matrix}d 1 \\ d 2\end{matrix} ]$$

wherein the entry d1 can be a class probability “fluorescence positive” and the entry d2 can be a class probability “fluorescence negative”. The second partial confidence measure can thus be such a vector d for an image subsection.

In step S5, on the basis of the second partial confidence measures ZTKM and the first partial confidence measures ETKM, which are entered in step S5, the confidence measure KM of the presence of the fluorescence pattern is determined. Preferably, in a step S6, the confidence measure KM is output. Such an output can preferably take place in the form of an output of a data element via a data interface or particularly preferably via an optical output on a display of a computing unit such as a computer monitor.

The confidence measure KM can preferably be determined in that for a respective subsection or a respective image subsection having index k=1 . . . K, a respective averaging of the respective first partial confidence measure and the respective second partial confidence measure takes place, so that respective averaged partial confidence measures for respective image subsections result. These averaged partial confidence measures can then be averaged once again over all image subsections or all indices k=1 . . . K in order to determine the confidence measure of the presence of the fluorescence pattern.

According to FIG. 5, alternatively to step S5, a modified step S51 having substeps S511 and S512 can be carried out. In step S511, the first partial confidence measures ETKM and the second partial confidence measures ZTKM are entered. In the first step, weighted partial confidence measures are determined via weighting of the first partial confidence measures ETKM and the second partial confidence measures ZTKM. The weighted partial confidence measures can be provided as a data element GTKM. The confidence measure KM is then determined in step S512 on the basis of the weighted partial confidence measures GTKM.

According to one preferred embodiment, a weighting thus takes place per image subsection of the first partial confidence measure and the second partial confidence measure. This can thus be designated as a determination of respective weighted partial confidence measures via weighting of the respective first partial confidence measures and the respective second partial confidence measures.

In one particular embodiment, a weighting thus takes place using weighting factors w1 and w2 per image subsection according to

$$\vec{r} = w 1 * \vec{o} + w 2 * \vec{d} = [ \begin{matrix}r 1 \\ r 2\end{matrix} ]$$

to ascertain one vector
\vec{rₖ}
   per image subsection k=1 . . . K.

These weighted partial confidence measures in the form of the vectors \vec{rₖ} per image subsection with index k=1 . . . K can then be used to determine the confidence measure KM. In particular, the values r1 per image subsection can preferably be observed which represent a class probability for the class “fluorescence positive”. These values R1 can then each be subjected to an application of a threshold value. The confidence measure is thus determined with the application of a threshold value to the weighted partial confidence measures.

An image subsection is then preferably assigned to the class “fluorescence positive” when the corresponding value r1 is greater than the threshold value. For example, the value R1 can be in the value range of [0 . . . 1] and a threshold value of 0.6 is applied.

Image subsections for which the value r1 does not exceed the threshold value are then classified as belonging to the class “fluorescence negative”.

So-called majority voting can then preferably take place. In this majority voting, the confidence measure can then be ascertained in particular as a “yes”/“no” statement, wherein the majority of the image subsections is decisive. Thus, if there are more image subsections for which the fluorescence pattern was recognized as present or for which the class “fluorescence positive” was recognized, the confidence measure is thus “yes”. The inverse applies for the possibility of the confidence measure “no”.

The advantage of the method of an application of a threshold value to the weighted partial confidence measures is that it can be set in the method from which confidence measure value the overall fluorescence image is assessed as positive as such or the expected fluorescence pattern is assessed as present. This is settable in particular in dependence on the individual weighted partial confidence measures of the image subsections.

On the basis of a resulting grid structure in conjunction with a so-called striding of a neural network, such as the neural network NN1, it is possible that such a first neural network NN1 determines multiple items of location information which indicate respective subsections which overlap. A spatial separation of subsections or image subsections, as in the case of the image subsections TBB1, TBB2, TBB3 from FIG. 2C, does not necessarily have to be provided. It can also occur that image subsections are determined which at least partially overlap in their area. An overlap of multiple subsections can result in particular which indicate an identical relevant subsection, for example, an identical islet, on the substrate. Two subsections are then preferably viewed to be overlapping when their degree of area overlap exceeds a predetermined value. For example, a degree of overlap in a value range from [0 . . . 1] can be determined and then an overlap can first be determined when the degree of overlap exceeds a threshold value, for example the value 0.2.

Such overlapping image subsections can in principle indicate an identical or similar relevant image subsection. It is therefore advantageous to select only one from such overlapping image subsections, since a specific relevant subsection of a cell substrate is only to be entered once in the later evaluation for determining the confidence measure and certainly not multiple times.

FIG. 6 shows a preferred embodiment of the method according to the invention for this purpose.

The sequence of steps S1 and S2 is identical to the embodiment from FIG. 4. In step S2, the items of location information LI, the first partial confidence measures ETKM, and here also the presence confidence measures OB are determined, in particular per relevant subsection. Step S2 is then followed by a step S21, in which it is determined whether the location information, given by the data element LI, indicates a set of multiple overlapping subsections. In particular, in step S21 this set is determined. This set of the overlapping image subsections and their location information can be provided as a set of location information MLI as a data element.

From the set of overlapping image subsections, that image subsection or that item of location information is retained which has the highest value of the presence confidence measure OB.

In a step S22, an item of location information of a specific image subsection from this set of overlapping subsections is then ascertained and retained. The items of location information of the other image subsections from this set of the overlapping image subsections are discarded. Furthermore, items of location information which do not indicate overlapping image subsections are retained. The retained items of location information are then the basis for further processing. The retained items of location information are represented as a data element VLI. On the basis of the retained items of location information VLI, it can thus be ensured that with overlapping image subsections which indicate an identical relevant area of the substrate only a specific subsection is entered in the final evaluation or the processing. It is thus ensured that a relevant cell substrate area is only entered once as an image subsection in the later evaluation and does not corrupt the result.

In step S3, the respective image subsections are then extracted from the fluorescence image, here on the basis of the remaining or retained items of location information VLI.

FIG. 7 shows preferred steps for determining a brightness measure of the presence of the fluorescence pattern. In a step S100, a set of image subsections is determined which have a presence of the fluorescence pattern. For example, on the basis of the weighted partial image confidence measures GTKM, a set of image subsections can then be determined, the partial image confidence measure GTKM or the value R1 of which exceeds a threshold value.

This set of image subsections or remaining image subsections can then be represented as a data element VTB.

In a step S101, the brightness measure HM is then ascertained, of the presence of the fluorescence pattern on the immunofluorescence image on the basis of the set of image subsections VTB which have a presence of the fluorescence pattern.

Thus in particular only image subsections are taken into consideration as the image subsections VTB which have a class decision “positive fluorescence”.

For these image subsections, in particular an analysis takes place using statistics of the pixel values in these image subsections.

A histogram of the brightness values of the pixels is created per image subsection and then that brightness value is ascertained at which a 0.8 quantile or at which 80% of the pixels have an intensity value which is greater than the 0.8 quantile. This value of the 0.8 quantile is then to be used as the partial brightness value of an image subsection. For all image subsections to be observed, the respective partial image brightness values are then preferably subjected to averaging. These brightness quantile values of the respective image subsections are thus preferably averaged. Alternatively, a median value determination can be carried out.

This brightness quantile value ascertained via median or mean value can then be output as the brightness measure HM.

Particularly preferably, the resulting brightness quantile value is further quantified in four quantification ranges. For example, the brightness quantile value can be in a value range from [0 . . . 255], so that a quantification takes place on four quantification ranges having step values from [1 . . . 5].

Such a step value can then be output to the user.

FIG. 8 shows a preferred structure of the second neural network NN2. The neural network NN2 analyses a respective partial image TB and determines a respective second partial confidence measure ZTKM for a respective partial image TB.

For this purpose, the neural network NN2 can make use of a sequence of respective convolutional modules CM. A respective convolutional module CM preferably comprises for this purpose a sequence of a convolutional step CS, a batch normalization step BS, a further convolutional step CS, and a further batch normalization step BS, followed by a max pooling step MAS.

Multiple such convolutional modules CM can follow one another.

The last convolutional module CM can preferably be followed by further processing via multiple dense layers DL, also called fully connected layers. The second partial confidence measure ZTKM can then preferably be determined via a softmax layer XML.

FIG. 9 shows a device V1, via which the method according to the invention can preferably be carried out. The device V1 can be designated as a fluorescence microscope. The device V1 comprises a holder H for a substrate S or an object carrier having such a substrate S, which was incubated in the above-described manner. Excitation light AL of an excitation light source LQ is conducted toward the substrate S via an optical unit O. Resulting fluorescence radiation FL is then transmitted back through the optical unit O and passes the dichroic mirror SP1 and an optional optical filter F2. The fluorescence radiation FL preferably passes an optical filter FG, which filters out a green channel. A camera K1 is preferably a monochromatic camera, which then captures the fluorescence radiation FL in a green channel if an optical filter FG is present. According to an alternative embodiment, the camera K1 is a colour image camera, which manages without using the optical filter FG and captures the fluorescence image in the corresponding colour channel as a green channel via a Bayer matrix. The camera K1 provides the image information VI or the fluorescence image to a computing unit R, which processes this image information B1. The computing unit R can preferably output or provide data ED, such as a fluorescence image and/or confidence measures, via a data interface DS1.

FIG. 10 shows a computing unit according to the invention, which, according to a preferred embodiment, preferably accepts a fluorescence image FB as a data signal S1 via a data interface DS2. The computing unit R can then ascertain the above-described items of information and provide them via a data interface DS3 as a data signal S13. This can preferably take place via a wired or wireless data network. The computing unit R particularly preferably comprises an output interface AS for outputting the information via an output unit AE. The output unit AE is preferably a display unit for an optical display of the above-mentioned information.

FIG. 11 shows a data network device DV according to the invention according to one preferred embodiment. The data network device DV accepts the fluorescence image FB as a data signal SI1 via a data interface DS4. The data network device DV comprises an above-described computing unit R and a memory unit MEM. The computing unit R, a memory unit MEM, and also the data interface DS4 are preferably connected to one another via an internal data bus IDB.

FIG. 12 shows an embodiment of a proposed computer program product CPP. The computer program product CPP can have its data signal SI2 accepted via a data interface DSX by a computer CO.

FIG. 13 shows a result table of results for the biological cell substrate in the embodiment of a cell smear of Crithidia luciliae.

4923 fluorescence images which, according to the ground truth information, have no fluorescence or are to be classified as “negative”, were also predicted as negative by the method according to the invention.

23 negative fluorescence images were classified as positive by the method according to the invention.

2532 fluorescence images having the fluorescence class “positive” were also classified by the method according to the invention as “positive”.

195 images of the fluorescence class “positive” were classified as the fluorescence class “negative” by the method according to the invention.

Depending on the specific implementation requirements, exemplary embodiments of the invention can be implemented in hardware or in software. The implementation can be carried out using a digital storage medium, for example, a floppy disc, a DVD, a Blu-ray disc, a CD, a ROM, a PROM, an EPROM, an EEPROM, or a FLASH memory, a hard drive, or another magnetic or optical memory, on which electronically readable control signals are stored which can interact or interact with a programmable hardware component in such a way that the respective method is carried out.

A programmable hardware component, for example, in particular a computing unit, can be formed by a processor, a computer processor (CPU=Central Processing Unit), a graphics processor (GPU=Graphics Processing Unit), a computer, a computer system, an application-specific integrated circuit (ASIC), an integrated circuit (IC), a one-chip system (SOC=System on Chip), a programmable logic element, or a field-programmable gate array having a microprocessor (FPGA).

The digital storage medium can therefore be machine-readable or computer-readable. Some exemplary embodiments thus comprise a data carrier which has electronically readable control signals which are capable of interacting with a programmable computer system or a programmable hardware component in such a way that one of the methods described herein is carried out. One exemplary embodiment is thus a data carrier (or a digital storage medium or a computer-readable medium), on which the program for carrying out one of the methods described herein is recorded.

In general, exemplary embodiments of the present invention can be implemented as a program, firmware, computer program, or computer program product having a program code or as data, wherein the program code or the data is or are active so as to carry out one of the methods when the program runs on a processor or a programmable hardware component. The program code or the data can also be stored, for example, on a machine-readable carrier or data carrier. The program code or the data can be provided, inter alia, as source code, machine code, or byte code and as other intermediate code.

A further exemplary embodiment is furthermore a data stream, signal sequence, or sequence of signals which represent or represents the program for carrying out one of the methods described herein. The data stream, the signal sequence, or the sequence of signals can be configured, for example, so as to be transferred via a data communication connection, for example, via the Internet or another network. Exemplary embodiments are thus also signal sequences representing data which are suitable for transmission via a network or data communication connection, wherein the data represent the program.

A program according to one exemplary embodiment can implement one of the methods during its performance, for example, in that it reads memory locations or writes one datum or multiple data therein, by which switching processes or other processes can possibly be induced in transistor structures, in amplifier structures, or in other electrical, optical, magnetic components or components operating according to another functional principle. Accordingly, by reading out a memory location, data, values, sensor values, or other items of information can be captured, determined, or measured by a program. A program can therefore capture, determine, or measure variables, values, measured variables, and other items of information by reading out one or more memory locations.

1. Method for detecting a presence of a fluorescence pattern on an immunofluorescence image of a biological cell substrate,
the method comprising
incubating the cell substrate (S) with a liquid patient sample, which potentially comprises primary antibodies, and furthermore with secondary antibodies, which are marked using a fluorescence stain, irradiating the cell substrate using an excitation radiation, and capturing the immunofluorescence image (FB),
determining respective items of location information (LI), which indicate respective locations of respective relevant subsections of the cell substrate in the fluorescence image, and determining respective first partial confidence measures (ETKM) of respective presences of the fluorescence pattern on the respective subsections using a first neural network (NN1) on the basis of the overall fluorescence image,
extracting respective image subsections (TB), which correspond to the respective subsections of the cell substrate, from the fluorescence image on the basis of the items of location information (LI),
determining respective second partial confidence measures (ZTKM) of respective presences of the fluorescence pattern on the respective subsections using a second neural network (NN2) on the basis of the respective image subsections (TB),
determining a confidence measure (KM) of the presence of the fluorescence pattern in the fluorescence image (FB) on the basis of the first partial confidence measures (ETKM) and the second partial confidence measures (ZTKM).

2. Method according to claim 1,
furthermore comprising outputting the confidence measure (KM).

3. Method according to claim 1,
furthermore comprising
determining weighted partial confidence measures (GTKM) via weighting of the first partial confidence measures (ETKM) and the second partial confidence measures (ZTKM),
determining the confidence measure on the basis of the weighted partial confidence measures.

4. Method according to claim 3,
furthermore comprising determining the confidence measure (KM) via application of a threshold value to the weighted partial confidence measures (GTKM).

5. Method according to claim 1,
furthermore comprising
determining respective presence confidence measures (OB), which indicate for the respective locations of the respective relevant subsections to which degrees at the respective locations actually relevant subsections of the cell substrate are present, using the first neural network on the basis of the overall fluorescence image,
determining whether the items of location information (LI) indicate a set (MLI) of multiple overlapping image subsections,
retaining the item of location information of a specific image subsection from the set of overlapping image subsections on the basis of the presence confidence measures (OB) of the overlapping image subsections and discarding the items of location information of the other image subsections from the set of overlapping image subsections,
extracting respective image subsections from the fluorescence image (FB) on the basis of the remaining items of location information (VLI).

6. Method according to claim 1,
furthermore comprising
determining a set of image subsections which comprise a presence of the fluorescence pattern,
ascertaining a brightness value (HM) of the presence of the fluorescence pattern on the immunofluorescence image on the basis of the set of image subsections which comprise a presence of the fluorescence pattern.

7. Method for digital image processing,
comprising
providing an immunofluorescence image (FB), which represents a staining of a biological cell substrate (S) by a fluorescence stain,
determining respective items of location information (LI), which indicate respective locations of respective relevant subsections of the cell substrate (S) in the fluorescence image (FB), and determining respective first partial confidence measures (ETKM) of respective presences of the fluorescence pattern on the respective subsections using a first neural network (NN1) on the basis of the overall fluorescence image (FB),
extracting respective image subsections (TB), which correspond to the respective subsections of the cell substrate, on the basis of the items of location information (LI),
determining respective second partial confidence measures (ZTKM) of respective presences of the fluorescence pattern on the respective subsections using a second neural network (NN2) on the basis of the respective image subsections (TB),
determining a confidence measure (KM) of the presence of the fluorescence pattern in the fluorescence image on the basis of the first partial confidence measures (ETKM) and the second partial confidence measures (ZTKM).

8. Computer program product (CPP), comprising commands which, upon the execution of the program by a computer, prompt it to carry out the method for digital image processing according to claim 7.

9. Data carrier signal (SI2), which transmits the computer program product (CPP) according to claim 8.

10. Device for detecting at least one fluorescence pattern on an immunofluorescence image (FB) of a biological cell substrate,
comprising
a holding device (H) for an object carrier having the cell substrate (S), which was incubated with a patient sample, including the autoantibodies, and furthermore with secondary antibodies, which are each marked using a fluorescence stain,
at least one image capture unit (K1) for capturing a fluorescence image (SG) of the cell substrate (S)
and furthermore comprising at least one computing unit (R), which is designed to execute the following steps
determining respective items of location information (LI), which indicate respective locations of respective relevant subsections of the cell substrate in the fluorescence image (FB), and determining respective first partial confidence measures (ETKM) of respective presences of the fluorescence pattern on the respective subsections using a first neural network (NN1) on the basis of the overall fluorescence image (FB),
extracting respective image subsections, which correspond to the respective subsections of the cell substrate, on the basis of the items of location information (LI),
determining respective second partial confidence measures (ZTKM) of respective presences of the fluorescence pattern on the respective subsections using a second neural network (NN2) on the basis of the respective image subsections,
determining a confidence measure (KM) of the presence of the fluorescence pattern in the fluorescence image (FB) on the basis of the first partial confidence measures (ETKM) and the second partial confidence measures (ZTKM).

11. (canceled)

12. Data network device (DV),
comprising at least one data interface (DS4) for accepting a fluorescence image (FB), which represents a staining of a cell substrate by a fluorescence stain,
and furthermore comprising at least one computing unit (R), which is designed to execute the following steps in the course of digital image processing
determining respective items of location information (LI), which indicate respective locations of respective relevant subsections of the cell substrate (S) in the fluorescence image (FB), and determining respective first partial confidence measures (ETKM) of respective presences of the fluorescence pattern on the respective subsections using a first neural network (NN1) on the basis of the overall fluorescence image (FB),
extracting respective image subsections (TB), which correspond to the respective subsections of the cell substrate, on the basis of the items of location information (LI),
determining respective second partial confidence measures (ZTKM) of respective presences of the fluorescence pattern on the respective subsections using a second neural network (NN2) on the basis of the respective image subsections,
determining a confidence measure (KM) of the presence of the fluorescence pattern in the fluorescence image on the basis of the first partial confidence measures (ETKM) and the second partial confidence measures.
